(19) United States
(12) Patent Application Publication
(10) Pub. No.: US 2007/0265693 A1
(43) Pub. Date: Nov. 15, 2007

(54) Coronary sinus catheter system and method

(76) Inventor: Larry D. Paskar, Chesterfield, MO (US)

Correspondence Address: HUSCH & EPPENBERGER, LLC, 190 CARONDELET PLAZA, SUITE 600, ST. LOUIS, MO, 63105-3441, US

(21) Appl. No.: 11/434,368

(22) Filed: May 15, 2006

(51) Int. Cl. A61N 1/00

(52) U.S. Cl. 607/122

(57) ABSTRACT

A coronary sinus catheter includes a pacing lead adapted for placement in a vein on the left ventricle of a heart having a right atrium and a coronary sinus. An outer member of the coronary sinus catheter has a distal end portion capable of having a curve formed remotely therein. The outer member is sized to be disposed in at least one chamber of said heart. An inner catheter has a passage therein for containing at least a portion of the pacing lead for placement thereof. The inner catheter has a distal end portion capable of having a curve formed therein. The distal end portions of the outer member and the inner catheter are disposable with respect to each other so that the distal end portions interact to allow cannulation of the coronary sinus irrespective of the orientation of the coronary sinus with respect to the right atrium of the heart.

### FIELD OF THE INVENTION

This invention relates to placement of pacing wires, and more particularly to placing a pacing wire in the coronary sinus and coronary veins.

### BACKGROUND OF THE INVENTION

Coronary sinus and coronary vein catheterization for placement of a pacing wire in the coronary veins has become an important new skill necessary for performing cardiac resynchronization therapy (CRT). Prior pacemakers or defibrillators required only one or two leads generally in the right atrium and/or the right ventricle. A relatively new three lead system allows heart failure patients who have dangerous electrical conduction problems of the heart to improve the heart's pumping efficiency while at the same time converting dangerous arrhythmias by its cardioverter-defibrillator function.

Congestive heart failure (CHF) is a very common and costly public health concern. Standard treatment relies on drugs which are often poorly effective, resulting in significant morbidity with poor quality of life, multiple hospitalizations, and an astoundingly high five year mortality of 50%.

The heart depends on a coordinated sequence of electrical impulse generation and conduction to allow appropriately timed atrial and ventricular chamber filling and contraction in order to optimize the efficiency of the heart's output, thereby maximizing its major function, which is pumping blood throughout the body.

Patients with heart failure may have conduction abnormalities. Drugs used to treat CHF can worsen these conduction abnormalities, further decreasing the output of a sick heart.

The three-lead CRT device serves to resynchronize the beating of the ventricles, resulting in coordinated and efficient ventricular contraction, thereby improving diastolic filling and systolic contraction, which results in improved cardiac blood output. Death from CHF may be due to progressive pump failure or arrhythmias resulting in sudden death. Biventricular pacing and resynchronization of the heart can decrease pump failure and, when combined with an implantable cardioverter-defibrillator (ICD), function to prevent sudden cardiac death. This is a very exciting and promising concept which deserves widespread application.

The third electrode placed through the coronary sinus into a coronary vein serves as a pacemaker for the left ventricle. That electrode runs along the left ventricular free wall, and allows for simultaneous pacing of both ventricles and more physiologic atrioventricular timing, which in turn results in more effective left ventricular contraction and subsequent improved cardiac blood output.

Coronary sinus/vein placement of a left ventricular lead is technically difficult at times, even for the most experienced electrophysiologist. Access to the venous system of the heart and particularly to the right atrium is carried out in a manner well-known in the art using standard, commonly practiced methods. Using these methods for accessing the coronary sinus, however, can pose difficulties. The position of the coronary sinus may vary significantly from patient to patient, especially in patients with diseased hearts and dilated heart chambers. Additionally, the catheterization under fluoroscopic guidance is carried out as the heart dynamically contracts and re-expands during the procedure. A great deal of time is now spent, even by highly skilled practitioners, locating the coronary sinus orifice and selectively catheterizing an appropriate branch vessel.

What is needed and heretofore has not existed is a system for accurately and reliably placing the left ventricular pacing lead under fluoroscopic guidance.

### SUMMARY OF THE INVENTION

Among the several objects and features of the present invention may be noted the provision of a coronary sinus catheter system and method which facilitates the accurate and reliable placement of pacing lead.

Another object is the provisions of a coronary sinus catheter system and method which provides a variable shape to facilitate use of the pacing lead.

Another object is the provision of a coronary sinus catheter system and method which may be reshaped in a body under imaging to accommodate the actual shapes of passages and/or spaces in the body to promote the placement and/or use of the pacing lead.

Other objects and features may become apparent in the detailed description of the invention below.

In a first aspect of the present invention, a coronary sinus catheter includes a pacing lead adapted for placement in a vein on the left ventricle of a heart having a right atrium and a coronary sinus, and an outer member having a distal end portion capable of having a curve formed therein. The outer member has apparatus for forming the curve in the outer member remotely and is sized to be disposed in at least one chamber of said mammalian heart. An inner catheter has a passage therein for containing at least a portion of the pacing lead for placement thereof, the inner catheter having a distal end portion capable of having a curve formed therein. The distal end portions of the outer member and the inner catheter are disposable with respect to each other so that the distal end portions interact to allow cannulation of the coronary sinus irrespective of the orientation of the coronary sinus with respect to the right atrium of the heart.

In a second aspect of the present invention, a method of cannulating a coronary sinus of a heart includes the steps of placing an outer member having a distal end portion capable of having a curve formed therein in the right atrium of the mammalian heart, and placing an inner catheter in the outer member. The outer member has structure for forming the curve in the outer member remotely and is sized to be disposed in said right atrium. The inner catheter has a distal end portion capable of having a curve formed therein. The method also includes disposing the distal end portions of the outer member and the inner catheter with respect to each other so that the distal end portions interact to allow cannulation of the coronary sinus irrespective of the orientation of the coronary sinus with respect to the right atrium of the heart.

### BRIEF DESCRIPTION OF THE DRAWINGS

FIG. 1 is a perspective drawing illustrating the use of the coronary sinus catheter system and method of the present invention in the human body.

FIG. 2 is a front elevation showing an illustrative first shape into which the coronary sinus catheter system may be formed in connection with its use with the pacing lead.

FIG. 3 is a front elevation showing an illustrative second shape into which the coronary sinus catheter system may be formed in connection with its use with the pacing lead.

FIG. 4 is a front elevation showing an illustrative third shape into which the coronary sinus catheter system may be formed in connection with its use with the pacing lead.

FIG. 5 is a front elevation showing an illustrative fourth shape into which the coronary sinus catheter system may be formed in connection with its use with the pacing lead.

FIG. 6 illustrates various possible up-going configurations of the coronary sinus catheter of the present invention.

FIG. 7 illustrates the basic out-of-plane shape achievable with the coronary sinus catheter of the present invention.

FIG. 7A illustrates various possible out-of-plane shapes of the coronary sinus catheter of the present invention.

FIG. 8 illustrates various possible down-going configurations of the coronary sinus catheter of the present invention.

Similar reference characters indicate similar parts throughout the several views of the drawings.

### DESCRIPTION OF THE PREFERRED EMBODIMENTS

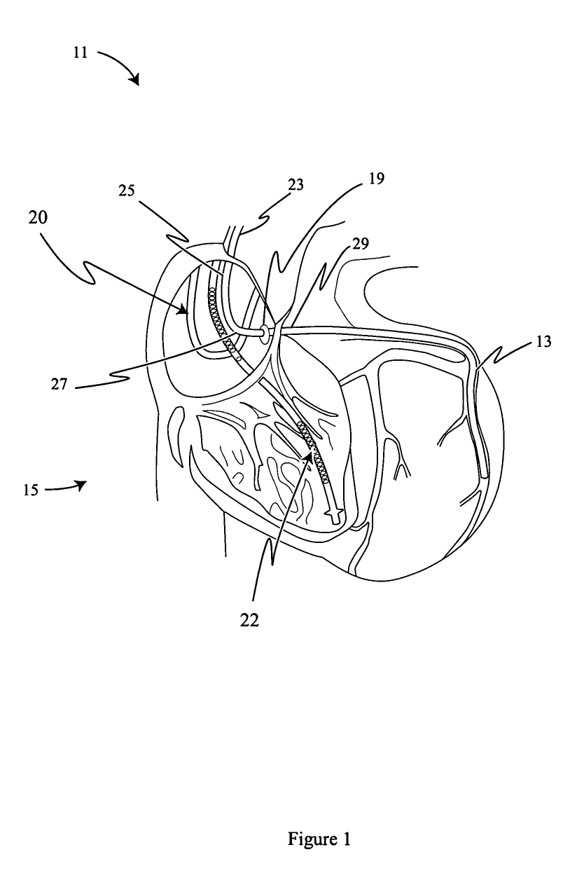

Turning now to the drawings, FIG. 1 shows the coronary sinus catheter 11 in place in the human body. (The particular shape of the coronary sinus catheter in FIG. 1 is illustrative only.) The catheter includes a pacing lead 13 adapted for placement in a vein on the left ventricle of a heart 15 having a right atrium 17 and a coronary sinus 19 for access to the vein disposed on the exterior of the left ventricle 21. Typically, such systems also include an atrial lead 20 and a right ventricle lead 22. The catheter itself has an outer member 23 having a distal end portion 25 capable of having a curve formed therein. It is preferred that the outer member be remotely controllable by a pull-wire or other conventional means to form the curve in the outer member remotely. As shown in FIG. 1, outer member 23 is sized to be disposed in at least one chamber of the mammalian heart 15. The catheter 11 also includes an inner catheter 27 having a passage therein for containing at least a portion of the pacing lead 13 for placement thereof. The inner catheter has a distal end portion 29 capable of having a curve formed therein. As will become apparent in connection with the description below, the distal end portions of the outer member and the inner catheter are disposable with respect to each other so that they interact to allow cannulation of the coronary sinus 19 irrespective of the orientation of the coronary sinus with respect to the right atrium of the heart.

Although from FIG. 1 one would conclude that cannulation of the coronary sinus is a straightforward process, that is not in fact the case. The orientations of the various parts of the heart shown in FIG. 1 are ideal and somewhat simplified. In practice, particularly with the diseased hearts for which the present invention is ideally designed, the placement and orientation of the coronary sinus is highly variable, and it is generally impossible to determine the exact shape that will be needed to cannulate the coronary sinus for placement of the pacing lead in the vein exterior of the left ventricle prior to actual insertion of the catheter and exploration. With conventional catheters, this process may require much experimentation and even the removal and replacement of the original catheter with a second (or even a third) catheter. All this exploration and replacement, of course, takes time and involves additional risk to the patient.

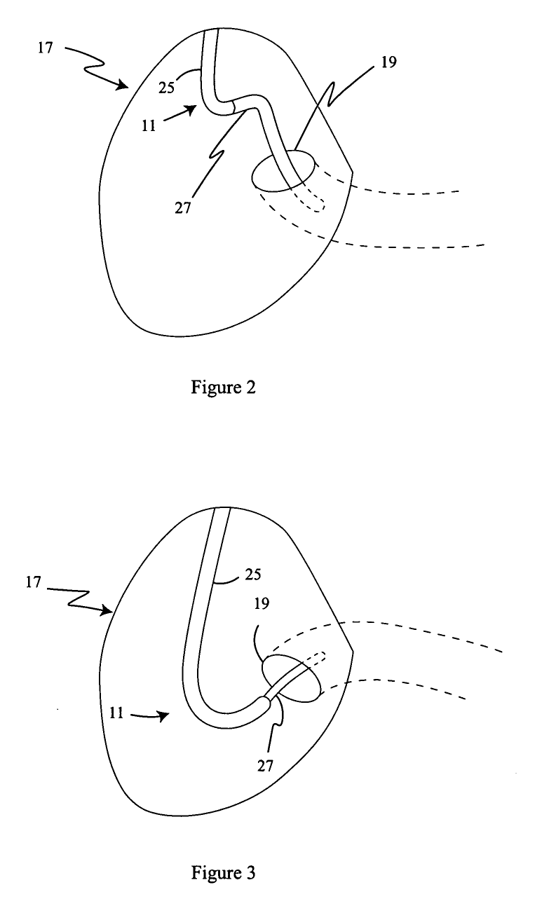

In FIG. 2 catheter 11 is formed into an up-going shape. To form this shape, the outer member and inner catheter are moved translationally with respect to each other and the distal ends thereof are formed in curves to make the desired shape of the catheter 11 to cannulate the coronary sinus, and held in that shape for use of that shape in the procedure. As can be seen, the orientation of the coronary sinus in this case varies substantially from the idealized situation shown in FIG. 1.

In FIG. 3 catheter 11 is formed into a down-going shape. Up-going and down-going in this application are defined by reference to the direction the outer member would move if it were moved further into the body. A curve pointing in the direction of further movement is up-going (FIG. 2), and a curve pointing opposite the direction of further movement is down-going (FIG. 3). In this shape, the catheter 11 may successfully cannulate the coronary sinus of FIG. 3 which opens into the right atrium in a very different direction from that of FIGS. 1 and 2.

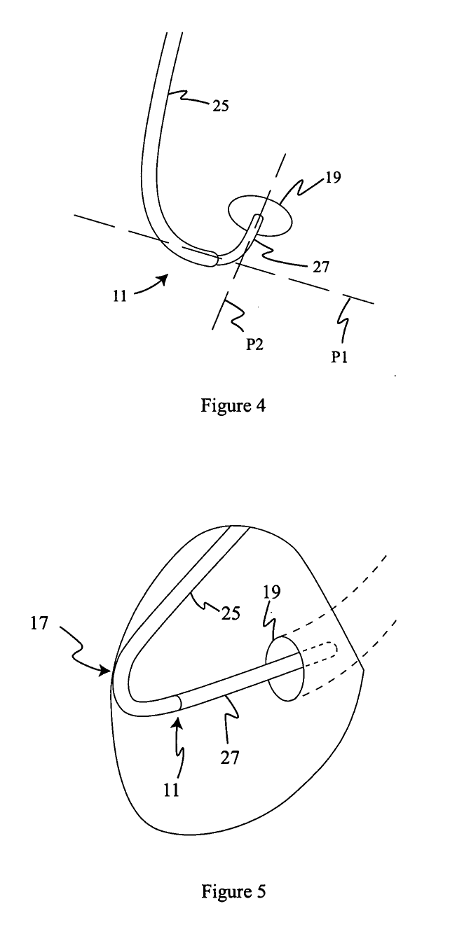

In FIG. 4 catheter 11 is formed into an out-of-plane shape. This is accomplished by curving the distal portion of outer member 25 to define a first plane P1 and rotating the curved distal end of inner catheter 27 so that it points along a second plane P2 which is not coincide with plane P1. Out of plane shapes are particularly useful in cannulating a coronary sinus that lies in a plane substantially outside the plane of the outer member.

Turning to FIG. 5, catheter 11 is formed into a shape such that it is wedged against the wall of the right atrium. This is a particularly useful configuration, since it provides a solid foundation for the catheter as a whole during placement of the pacing lead adjacent the left ventricle.

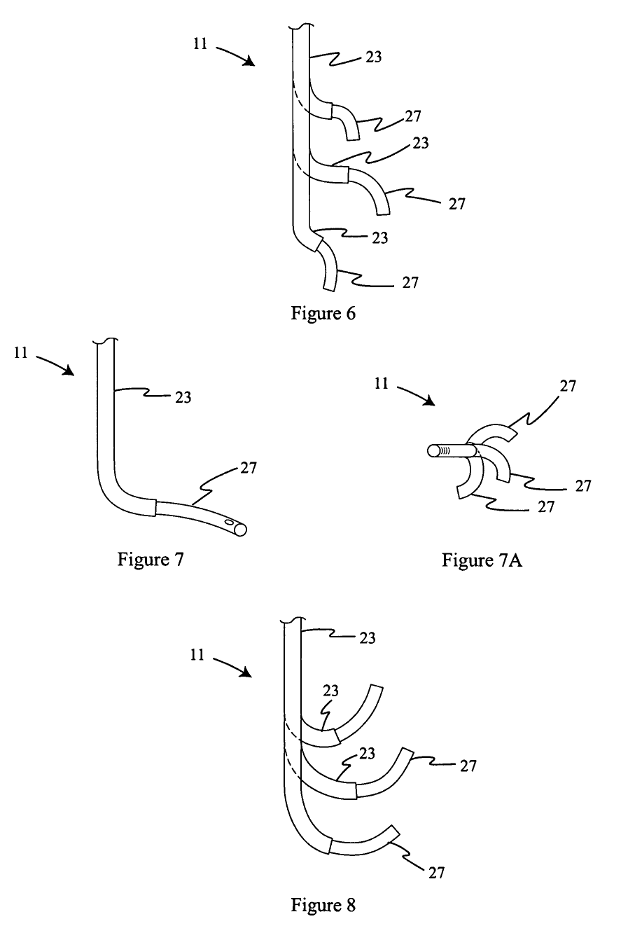

It should be appreciated that catheter 11 is capable of forming all the shapes of FIGS. 2-4 (and infinite variations thereof) with the same two elements-outer member 25 and inner catheter 27. By suitable translation and/or rotation of the outer member and inner catheter and adjustment of the curves in the distal end portions of each, an infinite number of shapes can be made, at least one of which may be used to successfully cannulate the coronary sinus. For example, FIG. 6 shows a few of the infinite number of up-going shapes that catheter 11 may be formed into to successfully cannulate the coronary sinus. Similarly, FIGS. 7 and 7A show a few of the infinite number of out-of-plane shapes that may be formed, and FIG. 8 shows a few of the infinite number of down-going shapes that may be formed.

Catheter 11 is particular useful in connection with accessing vessels or orifices that are in unusual positions. As human bodies age, the relative positions of various vessels and orifices change, so that a catheter of a conventional shape to enter that vessel may not be shaped correctly to do so.

That is, the inner catheter and the outer member interact to form the desired shape, whether it be up-going, down-going, out-of-plane, or any desired combination thereof. It is preferred that both the inner and outer members be capable of being imaged (such as by fluoroscopy or direct visualization, for example), so that the desired shape may be easily and readily achieved.

In view of the above, it will be understood that the various objects and features of the present invention are achieved and other advantageous results obtained.

What is claimed is:

1. A coronary sinus catheter comprising:
a pacing lead adapted for placement in a vein on the left ventricle of a heart having a right atrium and a coronary sinus;
an outer member having a distal end portion capable of having a curve formed therein which defines a plane, said outer member having means for forming said curve in the outer member remotely, said outer member being sized to be disposed in at least one chamber of said mammalian heart;
an inner catheter having a passage therein for containing at least a portion of the pacing lead for placement thereof, said inner catheter having a distal end portion capable of having a curve formed therein which defines a plane;
said distal end portions of the outer member and the inner catheter being disposable with respect to each other so that the distal end portions interact to allow cannulation of the coronary sinus irrespective of the orientation of the coronary sinus with respect to the right atrium of the heart.

2. The coronary sinus catheter as set forth in claim 1 wherein the outer member and inner catheter are adapted to interact to form an up-going shape to cannulate the coronary sinus, said outer member and inner catheter being imagable to disclose said shape to a human operator of the coronary sinus catheter.

3. The coronary sinus catheter as set forth in claim 1 wherein the outer member and inner catheter are adapted to interact to form a down-going shape to cannulate the coronary sinus, said outer member and inner catheter being imagable to disclose said shape to a human operator of the coronary sinus catheter.

4. The coronary sinus catheter as set forth in claim 1 wherein the outer member and inner catheter are adapted to interact to form an out-of-plane shape to cannulate the coronary sinus, said outer member and inner catheter being imagable to disclose said shape to a human operator of the coronary sinus catheter.

5. The coronary sinus catheter as set forth in claim 1 wherein the outer member and inner catheter are adapted to interact to place the distal end of the inner catheter in said coronary sinus.

6. The coronary sinus catheter as set forth in claim 1 wherein the outer member is adapted to form a stable platform for said coronary sinus catheter by forming a shape conforming with the internal anatomy of the right atrium.

7. The coronary sinus catheter as set forth in claim 1 wherein the outer member and inner catheter are adjustable to provide a second shape for accessing a branch vein from the coronary sinus irrespective of the orientation of the branch vein with respect to the coronary sinus.

8. A method of cannulating a coronary sinus of a heart, said mammalian heart having a right atrium, comprising the steps of:
placing an outer member having a distal end portion capable of having a curve formed therein in the right atrium of the heart, said outer member having means for forming said curve in the outer member remotely, said means being disposed at the proximal end of said catheter, said outer member being sized to be disposed in said right atrium;
placing an inner catheter in said outer member, said inner catheter having a distal end portion capable of having a curve formed therein;
disposing the distal end portions of the outer member and the inner catheter with respect to each other so that the distal end portions interact to allow cannulation of the coronary sinus irrespective of the orientation of the coronary sinus with respect to the right atrium of the heart.

9. The method as set forth in claim 8 wherein the distal end portions of the outer member and inner catheter interact to form an up-going shape to cannulate the coronary sinus, said outer member and inner catheter being imagable to disclose said shape to a human operator of the coronary sinus catheter.

10. The method as set forth in claim 8 wherein the distal end portions of the outer member and inner catheter interact to form a down-going shape to cannulate the coronary sinus, said outer member and inner catheter being imagable to disclose said shape to a human operator of the coronary sinus catheter.

11. The method as set forth in claim 8 wherein the distal end portions of the outer member and inner catheter interact to form an out-of-plane shape to cannulate the coronary sinus, said outer member and inner catheter being imagable to disclose said shape to a human operator of the coronary sinus catheter.

12. The method as set forth in claim 8 wherein the distal end portions of the outer member and inner catheter interact to place the distal end of the inner catheter in said coronary sinus.

13. The method as set forth in claim 8 wherein the outer member forms a stable platform for said coronary sinus catheter by forming a shape conforming with the internal anatomy of the right atrium.

14. The method as set forth in claim 8 wherein the outer member and inner catheter are adjusted to provide a second shape for accessing a branch vein from the coronary sinus irrespective of the orientation of the branch vein with respect to the coronary sinus.
